(19) United States
(12) Patent Application Publication
(10) Pub. No.: US 2006/0246570 A1
(43) Pub. Date: Nov. 2, 2006

(54) Isolation and use of decay fungi

(76) Inventors: Roberta Lee Farrell, Hamilton (NZ); Juan Javier Gonzalez Molina, Santiago (CL); Juan Eduardo Donoso Gutierrez, Santiago (CL)

Correspondence Address: JACOBSON HOLMAN PLLC, 400 SEVENTH STREET N.W., SUITE 600, WASHINGTON, DC, 20004, US

(21) Appl. No.: 10/554,052

(22) PCT Filed: Apr. 23, 2004
(86) PCT No.: PCT/NZ04/00078

§ 371 Date: Jan. 24, 2006

(30) Foreign Application Priority Data
Apr. 24, 2003 (NZ) 525521

(51) Int. Cl. D21C 3/00; C12N 1/14

(52) U.S. Cl. 435/278; 435/254.1

(57) ABSTRACT

The invention relates to methods for the isolation and development of fungi useful in the enhancement of wood or wood products quality and/or wood or wood products processing, and the decay fungi so developed. Compositions comprising or including decay fungi and methods for the use of said decay fungi and/or compositions are also provided. The decay fungi, compositions and methods are useful to provide improved cellulosic pulp production and/or wood and/or wood products of enhanced quality from non-sterilized wood.

This is a nationalization of PCT/NZ2004/000078 filed 24 Apr. 2004 and published in English.

### TECHNICAL FIELD

The present invention relates to the development of fungi useful in the enhancement of wood or wood products quality and/or wood or wood products processing. More particularly, but not exclusively, it relates to methods for the isolation and development of decay fungi, compositions comprising or including decay fungi and methods for the use of said decay fungi and/or compositions, said decay fungi, compositions and methods useful to provide improved cellulosic pulp production and/or wood and/or wood products of enhanced quality from non-sterilized wood.

### BACKGROUND

The pulp industry is based on separation of fibres, and the majority of pulp in the world is produced mainly by chemical processes of digestion. One example is the kraft system which uses wood, from natural forests or from plantations, such as Pinus radiata, and Eucalyptus spp as a raw material. This digestion process aims for a decrease in or an elimination of lignin, in order to obtain cellulosic pulps.

Such chemical digestion processes are frequently discussed due to the negative environmental impact caused by the resulting waste products and effluents and the contamination derived from them. For this reason, alternative processes of chemical digestion which have diminished environmental impact are being sought, such as processes which require less reagent, or processes which produce greater yield of cellulosic pulps with equivalent or decreased reagent and resulting effluent.

One route to a more environmentally favourable process is the use of biotechnology as part of the chemical digestion processes—for instance the application of decay fungi onto the wood substrate in order to have a more favourable digestion process and/or resulting cellulosic pulp.

Fungi are heterotrophic eukaryotic organisms which have enzymatic systems that allow them to depolymerize organic substances, previously synthesized by chlorophylic plants. In the field of the forest products pathology, decay fungi cause on wood a range of diverse alterations, such as molds, stains, and decay (hereinafter referred to as decay).

Three decay types are recognized on wood:
brown rot decay (holocellulose depolymerization) by means of enzymes that act via hydrolytic enzymatic action,
white rot decay (depolymerization of all the structural components of the woody cellular wall: mainly lignin and holocellulose, in secondary form), by means of hydrolytic and oxidative enzymes, and
soft rot decay by means of hydrolytic and oxidative enzymes which act on the substrate S2 of the secondary wall.

Lignin is a polymer of high complexity. It is a polyphenolic substance formed by three types of phenyl-propane units that form an aromatic heterogeneous compound and constitutes 20 to 30% of the cellular wall. Lignins, pectins, and holocellulose are the main structural and resistance elements of the cell wall. The biggest proportion of lignin is located in the middle lamella. Topographically, lignin is located mainly in the cellular vertexes, in form of units denominated G lignin (units of guacylics alcohols) that are highly condensed and of difficult chemical digestion.

Classic biopulping is defined as the treatment of wood with decay fungi prior to pulping, typically lignin degrading white-rot Basidiomycete fungi, in order to achieve the following:
Lignin degradation prior to conventional chemical digestion
Increase resultant effects on pulp and process—higher brightness, more strength improvements, more energy saving, more alkali savings.

Fungal delignification was first considered by West Virginia Pulp & Paper Co. (Westvaco) in 1957 and had the goal of inoculating chips with fungus during transport and storage for partial pulping. While the use of decay fungi for delignification of wood has been reported (See Erickson, K. E. and Kirk, T. K. “Biopulping, biobleaching and treatment of kraft bleaching effluents with white-rot fungi”. In: Comprehensive Biotechnology: the principles, applications and regulations of biotechnology in industry, agriculture and medicine. Pergamon Press, New York 271-294. (1985); Eriksson (1992); Blanchette R. A. et al., U.S. Pat. No. 5,476,790; Blanchette R. A. et al., U.S. Pat. No. 5,705,383; González M. J. et al., “Biopulping for Kraft Pulp of Pinus radiata”. Actas 50th Conference. APPITA, V, (1996); González M. J. et al., “Refining biokraft pulp of Radiata pine”. APPITA General Conference. APPITA, Melbourne, Australia, V (1997); González M. J. et al., “Biopulp from radiata Pine”. Actas 10th International Symposium on Wood and Pulping Chemistry. Yokohama, Japan, 06 (1999); González M. J. et al., “Yield increase with softwood Kraft Biopulp”. TAPPI Pulping/process & Product Quality Conference. Boston, USA. (2000); Donoso J. et al., “Influence of ecological factors in the behavior of White Decay Fungi”. Actas 53 APPITA Annual Conference. Rotorua, New Zealand, 03 (1999)) there has been no practical commercial application of the technology, mainly due to the non-competitive nature of the desired decay fungi, and also an unacceptably high level of cellulose degradation by the fungi. Thus there has been commercial limitation because in order to use the technology wood as a substrate, whether as log, chips, or shavings, requires decontamination, typically atmospheric steaming, in order to achieve a sterile, or in some circumstances almost sterile (that is, decontaminated) substrate, prior to fungal inoculation and supplementation with nutrients to stimulate fungal growth. This results in added costs, engineering requirements and logistics.

### OBJECTS OF THE INVENTION

It is an object of the present invention to provide a method of developing and/or using decay fungi to treat non-sterilized wood which will overcome the abovementioned disadvantages, and/or to provide decay fungi and/or a composition comprising or including decay fungi to be applied to non-sterilized wood is which subsequently to be treated by chemical process to obtain pulps, and/or methods of enhancing wood and/or wood products quality and/or wood processing utilizing said fungi and/or compositions, and/or which will at least provide the public with a useful alternative.

### SUMMARY OF THE INVENTION

In a first aspect of the present invention there is provided a method of isolating decay fungi which will have a positive effect on non-sterilized wood and/or wood products and/or wood processing by providing a lignocellulosic and/or extractives decrease in the wood and a minimisation or inhibition of the detrimental effects of competitor fungi, the method comprising or including the steps of:
1) collection of decay fungi (whether from nature or otherwise);
2) preparation of a cultivation of the decay fungi;
3) subjecting the cultivated decay fungi to a selection process to distinguish desired decay fungi from unwanted fungi, wherein the selection process includes or comprises subjecting the cultivated decay fungi to both:
   a) a test to establish production of oxidative enzymes; and
   b) a test to establish the ability of the cultivated decay fungi to outgrow and/or inhibit the development of competitor fungi,
and wherein the desired decay fungi will satisfy both tests;
4) isolation of the desired decay fungi.

In one embodiment, the method may additionally include or comprise the step of identifying the decay fungi at any time.

Preferably the decay fungi have a minimal deleterious effect on the wood and/or wood products and/or wood processing. In one example, the decay fungi have a minimal deleterious effect on cellulose yield and/or polymerisation.

In one embodiment, the collection of step 1) is from nature. In one example, the collection of step 1) is from soil and/or humus. In another example, the collection of step 1) occurs on wood and/or trees. In a further example, the collection of step 1) occurs on trees and/or wood of the species Pinus radiata and/or on trees and/or wood of Eucalyptus spp. The collection step can also be obtaining from a laboratory-maintained culture collection.

In various embodiments, the cultivation of step 3) occurs between particulate wood including wood chips, sawdust or the like as a solid cultivation, or in liquid supplemented growth media as a liquid cultivation, or in a combination of both as a semi-solid cultivation. In one example, the particulate wood of the solid and/or semi-solid cultivation is Pinus radiata or Eucalyptus spp.

In a second aspect of the invention there is provided a biologically pure culture of decay fungi which will have a positive effect on non-sterilized wood and/or wood products and/or wood processing by providing a lignocellulosic and/or extractives decrease in the wood and a minimisation or inhibition of the detrimental effects of competitor fungi isolated according to the above method.

In one embodiment, the decay fungi are of the class Basidiomycetes, order Aphyllophorales.

Preferably, said decay fungi may be selected from the genera comprising Pleurotus spp., Coriolus spp., Phanerochaete spp., Phlebia spp., Ganoderma spp., Lentinus spp.

Preferably, said fungi may be selected from the Pleurotus sp. strain 10-P or 24-P or the Coriolus sp. strain 15-A.

More preferably the decay fungi is a strain of Coriolus versicolor having all the identifying characteristics of the fungi of AGAL Accession Number NM02/32225.

In another embodiment, the decay fungi are of the class Ascomycetes, order Plectoascomycetes.

In a third aspect of the invention there is provided a biologically pure culture of decay fungi which when subjected to the steps of:
1) preparation of a cultivation of the decay fungi;
2) subjecting the cultivated decay fungi to a selection process to distinguish desired decay fungi from unwanted fungi, wherein the selection process includes or comprises subjecting the cultivated decay fungi to both:
   a) a test to establish production of oxidative enzymes; and
   b) a test to establish the ability of the cultivated decay fungi to outgrow and/or inhibit the development of competitor fungi,
and wherein the desired decay fungi will satisfy both tests;
3) isolation of the desired decay fungi;
   will, when applied to wood, have a positive effect on non-sterilized wood and/or wood products and/or wood processing by providing a lignocellulosic and/or extractives decrease in the wood and a minimisation or inhibition of the detrimental effects of competitor fungi.

Preferably the decay fungi have a minimal deleterious effect on the wood and/or wood products and/or wood processing. In one example, the decay fungi have a minimal deleterious effect on cellulose yield and/or polymerisation.

In a fourth aspect of the invention, there is provided a method for the preparation of a composition which will have a positive effect on non-sterilized wood and/or wood products and/or wood processing by providing a lignocellulosic and/or extractives decrease in the wood and a minimisation or inhibition of the detrimental effects of competitor fungi, the method comprising or including the steps of:
a) isolation of decay fungi as disclosed in the above method;
b) preparation of a reproductively viable form of said decay fungi;
c) use of said reproductively viable form of said decay fungi, optionally together with one or more acceptable carriers, diluents, or adjuvants, in the preparation of a composition.

In one embodiment, the isolation step a) of the method includes the preparation of the decay fungi on solid wood. In one example, said solid wood comprises raw wood residuals exemplified by but not limited to shavings, sawdust and/or chips. In another example, the solid wood is of the same genus and/or species as the wood for pulp production to which the composition is to be subsequently applied. In a further example, the solid wood is Pinus radiata or Eucalyptus spp.

Preferably the preparation of step b) of a reproductively viable form of the decay fungi is by massive vegetative reproduction.

In various embodiments, the preparation of step b) occurs between particulate wood including wood chips, sawdust or the like as a solid cultivation, or in liquid supplemented growth media as a liquid cultivation, or in a combination of both as a semi-solid cultivation. In one example, the particulate wood of the solid and/or semi-solid cultivation is Pinus radiata or Eucalyptus spp.

In another example, the preparation of step b) occurs between wood of the same genus and/or species as the wood for pulp production to which the composition is to be subsequently applied.

In one embodiment, the carrier is water (H₂O).

In a fifth aspect of the present invention there is provided a composition comprising decay fungi which will have a positive effect on non-sterilized wood and/or wood products and/or wood processing by providing a lignocellulosic and/or extractives decrease in the wood and a minimisation or inhibition of the detrimental effects of competitor fungi, prepared according to the above method.

In one embodiment, the composition is liquid. In an alternative embodiment, the composition is solid.

Preferably the decay fungi have a minimal deleterious effect on the wood and/or wood products and/or wood processing. In one example, the decay fungi have a minimal deleterious effect on cellulose yield and/or polymerisation.

Preferably, the decay fungi are, or the composition includes, Pleurotus sp. strain 10-P and/or 24-P and/or the Coriolus sp. strain 15-A.

It is presently most preferred that the decay fungi is, or the composition includes, Coriolus versicolor AGAL Accession Number NM02/32225.

In a sixth aspect of the present invention there is provided a method of enhancing wood or wood products quality, the method comprising or including the steps of:
a) preparation of a composition comprising or including decay fungi according to the above method;
b) application of the composition to non-sterilized wood subsequently used for pulp production.

In various embodiments the application of step b) of the composition may be manual and/or automated.

In various embodiments, the application in step b) of the composition is to the non-sterilized wood in the forest and/or storing yard and/or mill.

Preferably the composition is applied to the wood used for pulp production at a ratio of between about 0.05% and about 5% (w/w) decay fungi/dry weight of wood.

In one embodiment, the method may further include the step of maintaining the wood to which the composition has been applied under conditions which allow growth of the decay fungi for a term sufficient to allow a minimisation or inhibition of the detrimental effects of competitor fungi. In an alternative or additional embodiment, the method may further include the step of maintaining the wood to which the composition has been applied under conditions which allow growth of the decay fungi for a term sufficient to effect a lignocellulosic and/or extractives decrease in said wood. Preferably, the composition is applied so as to be in contact with the non-sterilized wood for a period of from about 4 days to about 4 months.

Preferably, the composition is applied to wood chips so as to be in contact with wood for a period of about 7 days.

Preferably, the composition is applied so that greater than 50% of the wood is colonized by the decay fungi.

Preferably, the decay fungi is, or the composition includes, Pleurotus sp. strain 10-P and/or 24-P and/or the Coriolus sp. strain 15-A.

It is presently most preferred that the decay fungi is, or the composition includes, Coriolus versicolor AGAL Accession Number NM02/32225.

In a seventh aspect of the invention there is provided the wood or wood products prepared according to the method described above.

In an eighth aspect of the present invention there is provided a method of improved chemical and/or modified chemical pulping, the method comprising or including:
a) preparing a composition comprising or including decay fungi;
b) applying the composition to non-sterilized wood to be used for pulp production; and
c) pulping said wood in a chemical and/or modified chemical pulping process.

Preferably the pulping of step c) is in a kraft and/or modified kraft process

In one embodiment the preparation of step a) of a composition comprising or including decay fungi is by a method as herein disclosed.

In various embodiments the application of step b) of the composition provides an increase in pulping efficiency, in creased yield, and/or lower kappa numbers.

Additionally or alternatively the application of step b) of the composition provides a reduction in pulping energy consumption. Additionally or alternatively the application of step b) of the composition provides a reduction in pulping chemical processing liquour consumption.

Preferably the application of step b) of the composition of step b) may be manual and/or automated.

Preferably in step b) the composition is applied to non-sterilized wood which comprises logs with or without bark and/or chips.

In various embodiments, in step b) the composition is applied to non-sterilized wood used for pulp production in the forest and/or storing yard and/or mill.

Preferably in step b) the composition is applied to the non-sterilized wood at a ratio of between about 0.01% and about 5% (w/w) fungi/dry weight of wood, preferably between about 0.1% and about 1%, preferably less than about 1%, more preferably about 0.5% (w/w) fungi/dry weight of wood.

Preferably in step b) the composition is applied to non-sterilized wood which has a moisture content of from about 60% to about 80%.

Preferably in step b) the composition is applied to non-sterilized wood which comprises logs with or without bark and/or chips.

In one embodiment, the method may further include the step of maintaining the wood to which the composition has been applied under conditions which allow growth of the decay fungi for a term sufficient to allow a minimisation or inhibition of the detrimental effects of competitor fungi. In an alternative or additional embodiment, the method may further include the step of maintaining the wood to which the composition has been applied under conditions which allow growth of the decay fungi for a term sufficient to effect a lignocellulosic and/or extractives decrease in said wood. Preferably, the composition is applied so as to be in contact with the non-sterilized wood for a period of from about 4 days to about 4 months.

Preferably, the composition is applied to wood chips so as to be in contact with wood for a period of about 7 days.

Preferably, the composition is applied so that greater than 50% of the wood is colonized by the decay fungi.

Preferably, the decay fungi is, or the composition includes, Pleurotus sp. strain 10-P and/or 24-P and/or the Coriolus sp. strain 15-A.

It is presently most preferred that the decay fungi is, or the composition includes, Coriolus versicolor AGAL Accession Number NM02/32225.

In a ninth aspect of the invention there is provided a pulp prepared according to the method described above.

In a tenth aspect of the invention there is provided a biologically pure culture of Coriolus versicolor AGAL Accession Number NM02/32225.

In an eleventh aspect, the present invention relates to the use of Coriolus versicolor AGAL Accession Number NM02/32225 in a composition, method, or process of the invention.

### DESCRIPTION OF THE FIGURES

The invention will now be described with reference to the Figures in which:

FIG. 1 presents a wood cut used to reproduce the decay fungi

FIG. 2 illustrates the development of the fungi on a specific selected substrate;

FIG. 3 illustrates decay fungi sample development and antagonism/antibiosis results at different temperatures;

FIG. 4 illustrates oxidative enzyme production by various fungi;

FIG. 5 presents a sample of the preferred decay fungi action on the fibres of cell corners and mediates.

### DETAILED DESCRIPTION OF THE INVENTION

The present invention provides a method for the development of decay fungi which comprises or includes the collection, isolation and selection of decay fungi and the use of said decay fungi in the preparation of a composition. The invention further provides methods to improve cellulose pulp production via chemical processes, for example, kraft process and/or sulfite pulp process, through the use of decay fungi and/or a composition comprising or including decay fungi wherein said decay fungi are capable of providing delignification and/or an extractives decrease in non-sterilized wood.

As used herein “fungi” may refer to more than one strain of fungi or to a single strain of fungi as will be apparent from the context.

As used herein “non-sterilized wood” means wood and/or wood products that have not been subjected to a sterilization or other pretreatment process.

As used herein “extractives” means the collective components of wood that can be extracted by organic solvents, including triglycerides, fatty acids, resin acids, sterols, waxes, and unsaponifiable compounds. Wood typically contains from 1-4% extractives on a dry weight basis, and up to 300 or more specific chemical compounds are present in the collective term “extractives”.

As used herein “outgrow” means the ability of a decay fungi to proliferate more rapidly than a competitor and/or colonize a greater proportion of substrate than a competitor. This can be demonstrated in, for example, a plate assay wherein cultivated fungi will dominate suitable media when presented with competitive fungi.

As used herein “biopulp” means a cellulose pulp derived from wood to which decay fungi of the invention has been applied.

As used herein “competitor fungi” means any fungi that is not a decay fungi of the present invention.

The present invention recognizes that in order to be capable of providing a delignification and/or extractives decrease in non-sterilized wood, the decay fungi of the present invention must be competitive with and preferably out compete competitor unwanted fungi. The ability of the decay fungi to compete with and/or outgrow competitor fungi allows the decay fungi to colonize a substantial proportion of, and preferably the majority of, the wood substrate to which it is applied, thereby to achieve a delignification and/or extractives decrease and/or other benefits attributable to a colonization of the wood source by the decay fungi in a substantial proportion of said wood. Such additional benefits include but are not limited to a minimization of the detrimental effects of competitor fungi and/or other organisms on the wood source. For example, unwanted competitor fungi may produce enzymes that degrade cellulose and would, if able to colonize a substantial proportion of the wood source, thereby lead to a decrease in cellulose yield. Application of competitive decay fungi of the present invention capable of minimizing the deleterious effects of unwanted competitor fungi may thereby enhance the cellulose pulp production of, and/or enhance the wood products quality derived from, a wood source.

The invention further provides techniques useful in the selection of decay fungi, to identify and select for desirable characteristics of the decay fungi thereby to optimise the action of decay fungi. These desirable characteristics include the main depolymerization activity of the decay fungi being against lignin, and the ability of the decay fungi to compete with, and preferably outgrow, competitor fungi.

The lignin depolymerization activity of the fungi is dependant on enzymes, such as oxidative enzymes, such as laccase, manganese (Mn) peroxidase and lignin peroxidase.

Production of oxidative enzymes can be determined by any method known in the art. The presence of detectable extracellular lignin peroxidase and/or manganese-dependant peroxidase and/or laccase in a cell-free assay is indicative of exemplary oxidative enzyme production by decay fungi. Preferred decay fungi of the present invention will be capable of producing greater than about 0.005 International Units oxidative enzyme activity per mL of growth media in such a cell-free assay.

The present invention recognizes the effect of environmental variables on the behavior of decay fungi, and in particular, on the production of oxidative enzymes and resistance of the decay fungi to competitor fungi.

Many factors influence the overall efficiency of the decay process of wood, and the period for which the wood is exposed to the decay fungi to, for example, observe a delignification and/or extractives decrease. These factors include: physical environmental factors, such as moisture content and temperature; chemical factors, for instance pH or the concentration of oxygenate or carbonic anhydride; biological factors, for instance the wood species, or the species of competitor fungi.

The present invention recognizes the effect of environmental factors on the growth and function of decay fungi to provide enhanced wood and/or wood products quality and/or wood processing, and the importance of environmental factors to the application of decay fungi to non-sterilized wood.

The decay fungi of the present include species of Basidiomycetes, order Aphyllophorales, and species of Ascomycetes, order Plectoascomycetes.

The decay fungi of the present invention may be collected from nature, or may be obtained from repositories, such as the Australian Government Analytical Laboratories (AGAL) or the American Type Culture Collection (ATCC).

Methods for the isolation and culturing of fungi are well known in the art. Three general means of fungal inocula preparation and culturing exist:
a) liquid—cultivation and propagation of conidia and/or spores and/or somatic body (hyphae) in nutritious liquid solutions;
b) semi-solid—cultivation and propagation utilizing a solid material diluted thoroughly in nutritious liquid solutions; and
c) solid—cultivation and propagation utilizing a solid, relatively humidified material.

Liquid cultivation has been broadly used. Semi-solid cultivation with respect to methods of the present invention shows little diffusion of the fungi onto the wood. Also, liquid cultivation and semi-solid cultivation utilize a support enriched with nutrients which can allow the colonization of, many competitor fungi, rendering liquid and semi-solid cultivation methods ill-suited to use in uncontrolled atmospheres, such as in a forest or mill, and particularly so on non-sterilized wood.

The present invention provides fungi, compositions, and methods in part for the delignification of solid wood or chips. As shown in Example 4 herein, decreasing lignin present in the wood by treatment with decay fungi of the invention decreases the chemical digestion required to process the wood to pulp, minimizing unfavorable environmental impacts and improving pulping process efficiency. As can be seen in Example 2 herein, treatment of wood with the decay fungi, compositions, and methods of the invention also enhances the quality of wood products, for example, paper quality.

Thus the present invention also relates to the application to wood, such as logs and/or chips of softwoods and hardwoods (specifically illustrated by examples of Pinus radiata or Eucalyptus spp), of compositions comprising or including at least one decay fungi, with the main objective being the digestion of a component of the cellular wall, especially lignin, thereby reducing subsequent chemical cooking requirements (either amount of chemical, time or temperature) in the industrial production of cellulose pulp. This provides improved yields, diminished requirements for bleaching reagents, easier refining of fibers for wood products and particularly paper production, and wood products of improved properties and quality (for example, improved tear indices and tensile energy absorption (TEA) of paper).

The methods of the invention consider the application of a composition comprising or including decay fungi to non-sterilized wood subsequently to be pulped in a chemical and/or modified chemical pulping process. The wood to which the decay fungi are applied may be in any solid form, typically logs with or without bark and/or chips. The decay fungi compositions and methods of the present invention may be used in the processing of wood of any species, and in particular but not limited to the processing of wood of Pinus radiata and Eucalyptus spp.

The application of the decay fungi and/or the composition comprising or including decay fungi can be directly in the forest, or mills or at any location during the processing of the wood, including during transportation of the wood and/or wood products. The intensity of application of the decay fungi and/or composition comprising or including decay fungi can vary, and may at any ratio from about 0.01% to about 5% weight of decay fungi to dry weight of wood.

The period for which the wood is treated with the fungi and/or composition of the invention varies from about 4 days to about 4 months, and is dependent upon the bioclimatic situation, the treatment conditions, the wood species and/or the quality of the product, and the form of the wood, for example, whether the wood comprises logs with or without bark, chips, and so on. As shown in Example 4 herein, a substantial enhancement in wood products quality and wood processing was achieved with the application of decay fungi to Pinus radiata logs wherein the decay fungi was in contact with the wood for 85 days, at a ratio of 0.6% (w/w) fungi/dry weight of wood.

The enhancement of wood and/or wood products quality and/or wood processing is achieved in part by the delignification by means of:
the isolation and development, for example between solid wood residuals, such as chips, shavings and/or sawdust of softwood and/or hardwood including but not limited to Pinus radiata or Eucalyptus spp, of decay fungi;
the use of the decay fungi in the preparation of a composition suitable for application to wood;
the application of the composition on the non-sterilized wood to be used later for cellulose pulp production,
whereby the application of the composition comprising or including decay fungi results in the digestion of at least part of the lignocellulose fraction in the wood.
The invention is further illustrated by the following non-limitative examples.

### EXAMPLE 1

A non-limitative application describing the procedure for obtaining and developing a decay fungi example of the invention is presented below.

The procedure includes the following steps:
a) Collection of the fungi in nature, whereby fungal material is isolated in form of sexual bodies from infected lignocellulosic matter. The fungi is identified later in the laboratory by the use of appropriate taxonomic key;
b) Isolation and cultivation of the decay fungi in growth medium. For example, mixtures of Agar and of Extract of Malt, more specifically between 1-3% Agar and 2-15% Extract of Malt is used as growth media, and may be supplemented with antibiotics such as penicillin and streptomycin;
c) Application of any standard method, such as the Nobleman method, to determine the presence of oxidative enzymes;
d) Determination of the effect of antibiosis exhibited by the fungi;
e) Preparation of the nutritious support for fungal multiplication;
f) Large-scale vegetative reproduction of the selected fungi.

### EXAMPLE 2

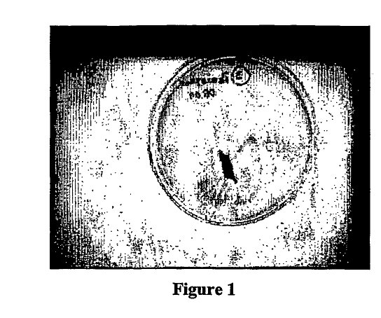

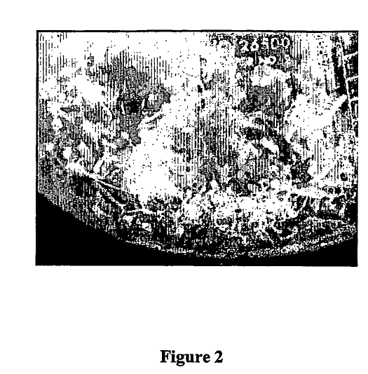

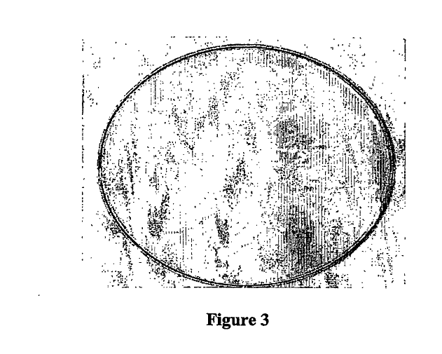

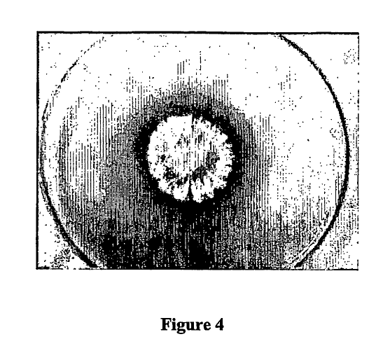

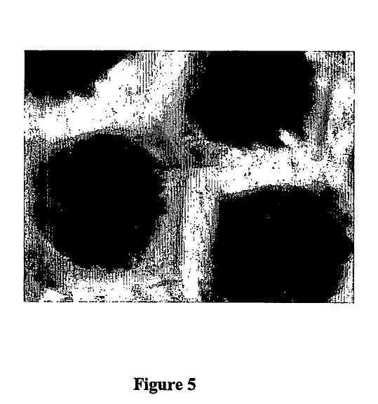

The following non-limitative example of the invention is presented to illustrate the steps of the inventive process of the invention, the results of the application of decay fungi in the processes of delignification of pulpwood, and the results of the final product of the pulping process.
a) Isolation: wooden pieces-with decay fungi are presented principally in cultivation of Agar-Malt with addition of bacterial antibiotics as is illustrated in FIG. 1. Once mycelia formation is achieved, the fungi is purified by standard microbiology techniques.
b) Preparation of a reproductively viable form and amount of decay fungi between solid wooden residuals: wooden residuals as chips, chips and/or sawdust of the forest species to be subsequently treated in the predelignification process with a variable moisture content between about 60% to about 80%, in either a natural state or previously sterilized, are inoculated with the selected decay fungi, as is shown in FIG. 2.
c) Tests of antibiosis, in the presence of competitive organisms: the decay fungus elect is cultivated and contacted or without contact but in the vicinity with a competitor fungi which is commonly present in the natural environment. This test is performed at different temperatures, as is illustrated in FIG. 3. The results and later analysis allow determination of the environmental factors under which the decay fungi provides the highest resistance to competition, antagonisms and/or undesired parasitisms.
d) Tests of oxidative enzymes: Cultivation of the decay fungi on an Agar substrate supplemented with tannins is used to determine the production of oxidative enzymes by the decay fungi is determined, wherein the level of enzyme production and resulting oxidation is revealed by the intensity of the brown coloration and the radius of the aureole surrounding the fungi, as illustrated in FIG. 4. The effect of temperature on enzyme production is determined by cultivating the decay fungi at different incubation temperatures.
e) Application of the composition comprising decay fungi on wood: Once grown on the substrate (sawdust, chip and/or splinters, etc), the decay fungi goes through a period of maturation in the laboratory of approximately 1 day to about 5 weeks, after which the composition can be applied in quantities of about 0.01% to about 5% (w/w fungi/wood) over industrial logs or chips of softwoods or hardwoods such as Pinus radiata or Eucalyptus spp.
f) Lignin removal in the woody cell: In FIG. 5 the oxidation of lignin is shown, particularly in the middle lamella, as observed in the cellular vertices where the largest quantity of highly condensed lignin exists.

Table I below presents the effect of environmental factors on oxidative enzyme production (in Units of enzyme accumulated in extracellular growth medium/ml) for five decay fungi isolated and developed as described above.

TABLE I

| | | | |
|---|---|---|---|
| | | | Mn peroxidase |
| Fungi isolate | Temperature | Laccase (U/ml) | (U/ml) |

| | | | |
|---|---|---|---|
| 9-C | 20° C. | 0.080 | 0.160 |
| | 25° C. | 0.130 | 0.120 |
| | 30° C. | 0.140 | 0.110 |
| 10-P | 20° C. | 1.500 | 0.240 |
| | 25° C. | 1.000 | 0.120 |
| | 30° C. | 0.260 | 0.020 |
| 24-P | 20° C. | 0.410 | 0.026 |
| | 25° C. | 1.810 | 0.063 |
| | 30° C. | 1.670 | 0.073 |
| 9-P | 20° C. | 1.770 | 0.320 |
| | 25° C. | 0.220 | 0.031 |
| | 30° C. | 1.890 | 0.250 |
| 15-A | 20° C. | 0.120 | 0.043 |
| | 25° C. | 0.060 | 0.028 |
| | 30° C. | 0.270 | 0.051 |

The decay fungi presented in Table I are species of Basidiomycetes, order Aphyllophorales, and are identified by the Applicant's isolate designations. The fungi were isolated and developed under different environmental development conditions, such as, for example, temperature, pH, concentration of carbonic anhydride, and the like. The results, expressed as concentration of oxidative enzymes, demonstrate the influence of temperature as a selection variable.

Table II below presents data on the characteristics of bleached pulps derived from pinus radiata wood to which the various decay fungi strains had been applied. These pulps were produced using the kraft process.

TABLE II

| | | | Kappa | | |
|---|---|---|---|---|---|
| | Yield % | Kappa | after O₂ | Delta Kappa | Yield after O₂ % |
| Control | 47.1 | 29.4 | 14.0 | 52.4 | 96.1 |
| 9 P | 49.5 | 31.5 | 21.1 | 33.0 | 97.5 |
| 9 C | 49.2 | 31.8 | 20.8 | 34.5 | 97.8 |
| 24 P | 49.8 | 30.7 | 20.9 | 31.9 | 99.0 |

| | Act. Chlor. CD | Viscos. mPa s | Yield % | Brightness | Total Cl |
|---|---|---|---|---|---|
| Control | 0.3 | 9.7 | 91.3 | 89.6 | 2.8 |
| 9 P | 0.2 | 16.7 | 92.5 | 92.6 | 2.8 |
| 9 C | 0.2 | 15.7 | 92.1 | 92.0 | 2.79 |
| 24 P | 0.2 | 18.5 | 94.0 | 92.6 | 2.78 |

The term kappa is a value resulting from a standard assay which is indicative of the quanitity and characterization of the residual lignin in kraft pulp. Yield is expressed in dry weight of pulp relative to the dry weight of wood entering the pulping process. The kappa after oxygen bleaching stage (O₂) corresponds to the value of the index after having submitted the pulp to delignification with O₂. Delta Kappa corresponds to the reduction in kappa following delignification by O₂. Yield after O₂ is as above but expressed in dry weight of O₂ bleached pulp over dry weight of the raw pulp as it entered the O₂ stage in %.

The control is the natural wood of the same species, in this case Pinus radiata, which was not treated with decay fungi.

As is shown in Table II, the pulp yield from wood treated with decay fungi is 5-6% more than in the control. With smaller rates of chlorine treatment or equivalent chlorine treatment (or any other oxidative bleach chemical), the samples of cellulose pulps derived from fungal treated wood have better yield after the process of bleaching. Pulps derived from fungi treated wood also exhibit higher final brightness, 3-5% higher than control.

The physical-mechanical properties of bleached papers produced from pulps derived from fungi treated wood (“biopulps”), and control pulps, are shown in Table III.

TABLE III

| | | | Tear I. | | | |
|---|---|---|---|---|---|---|
| | PFI | Freeness | (mNm²/ | Tensile | T.E.A. | Brightness |
| PULP | REV | (CSF) | g) | (kpa) | (j/kg) | (ISO) |
| BP-CH 10D | 1000 | 684 | 14.20 | 71.6 | 1309 | 87.7 |
| | 2000 | 657 | 11.28 | 85.8 | 1638 | 84.0 |
| | 4000 | 549 | 09.74 | 101.7 | 2100 | 83.3 |
| | 8000 | 260 | 08.46 | 110.6 | 2260 | 78.6 |
| BP-CH11B | 1000 | 651 | 10.76 | 75.8 | 1779 | 85.4 |
| | 2000 | 631 | 9.30 | 90.0 | 2095 | 84.6 |
| | 4000 | 542 | 8.10 | 102.5 | 2308 | 84.4 |
| | 8000 | 309 | 7.71 | 111.0 | 2506 | 82.4 |
| BP-CH12A | 1000 | 694 | 12.30 | 78.2 | 1596 | 87.1 |
| | 2000 | 663 | 9.72 | 93.2 | 1973 | 86.1 |
| | 4000 | 554 | 8.92 | 102.7 | 2218 | 84.4 |
| | 8000 | 308 | 7.98 | 112.1 | 2357 | 83.1 |
| CONTROL | 1000 | 630 | 8.58 | 59.5 | 1220 | 84.9 |
| | 2000 | 567 | 7.33 | 70.2 | 1458 | 83.9 |
| | 4000 | 415 | 6.15 | 78.7 | 1562 | 82.0 |
| | 8000 | 135 | 5.34 | 87.0 | 1740 | 76.6 |

The term Freeness, as measured by CSF units, corresponds to the measure of the capacity of pulp for retaining water. Tear Index (Tear I.) indicates the resistance of paper to rupture at the border of paper. Tensile indicates the resistance of paper to traction or tension. TEA corresponds to tensile energy absorption, that is resistance expressed by the internal cohesion of paper fibres. Brightness is the capacity of paper to reemit the light that impacts on paper, and is measured by standard Units called ISO.

The term PFI REV corresponds to various refining levels. For all the refining levels, the properties of papers derived from biopulps (BP—CH IOD, BP—CHIIB, BP—CH12A) are improved with respect to the control samples. The freeness of pulps is also improved. The tear indices for paper derived from each biopulp is better, by at least 38% compared to control. The tensile indices of paper derived from biopulps are greater by 24-36% compared to control. Biopulp TEA indices are also increased by 12-18%. The brightness of paper derived from biopulps is 2-4% higher compared to control.

### EXAMPLE 3

The following example presents data from a trial conducted on Radiata pine pulp using decay fungi in order to determine the bleaching capacity of the decay fungi. Additionally, the delignification of the Radiata pine wood treated with decay fungi before the Kraft pulping was observed.

Unbleached Radiata pine pulps at 28, 32, 35 and 42 Kappa Index were treated with two strains of decay fungi, strains 9C and 24P. Treatment was made at 0.06% decay fungi dry weight over pulp dry weight, and pulps were maintained at pH 6 at a temperature of 25° C. Decay fungi application was made on Bristol-board stage pulp and later the pulp was disaggregated and again taken to 70% moisture content. After treatment was concluded, pulps were disaggregated and ≈60 g/m2 paper sheets were prepared to allow a determination of the paper processing properties. Measurements of Kappa Index, viscosity, rupture length, tear index, brightness and opacity were made at 15, 25 and 35 days of treatment.

These measurements were made according to the following standards: Kappa Index T236 cm-85; Viscosity T230 om-89; Rupture length T404 om-87; Tear index T414 om-88; Brightness T452 om-92; and Opacity T425 om-91.

As shown below, treatment with either decay fungi on pulp at different Kappa levels resulted in a decrease in Kappa values. Even at low rates of residual lignin, the decay fungi had a delignification effect.

As shown in Table IV below, treatment of pressed Kraft pulp with decay fungi 9C causes a 30% decrease in Kappa index at 35 days of treatment. At the same time point, viscosity is 17% of the initial value. Rupture length drops to 50% of the control value and tear drops to 60% of the control value. Brightness decreases 12% and opacity increases 4%. As the Kappa Index increases, the delignification effect increases and the loss of properties decreases.

TABLE IV
Effect of treatment with decay fungi 9C on properties of pressed pulp

| | Kappa 35 | | | | Kappa 42 | | | |
|---|---|---|---|---|---|---|---|---|
| | Conrol | 15 days | 25 | 35 | Control | 15 days | 25 | 35 |
| Kappa | 35 | 32.6 | 30.3 | 24.4 | 42 | 38.2 | 27.7 | 24.6 |
| Viscosity | 1510 | 520 | 520 | 230 | 2260 | 730 | 410 | 210 |
| Tensile | 2420 | 1570 | 1260 | 1120 | 1820 | 950 | 1280 | 1360 |
| Tear | 1030 | 780 | 660 | 640 | 740 | 680 | 690 | 650 |
| Brightness | 27.1 | 26.1 | 24.0 | 26.2 | 23.6 | 24.5 | 22.1 | 24.1 |
| Opacity | 92.6 | 94.1 | 93.8 | 94.5 | 93.2 | 95.5 | 94.5 | 93.6 |

As shown in Table V below, treatment of disaggregated pulp with decay fungi 9C results in a Kappa loss of 20%, and a decrease in viscosity to 35% of control. There is a gain in brightness of 4% for Kappa 28, and 11% for Kappa 32 relative to control.

TABLE V
Effect of treatment with decay fungi 9C on properties of disaggregated
pulp

| | Kappa 28 | | | | Kappa 32 | | | |
|---|---|---|---|---|---|---|---|---|
| | Control | 15 days | 25 | 35 | Control | 15 days | 25 | 35 |
| Kappa | 28 | 23.6 | 22.8 | 22.6 | 32 | 29.5 | 28.3 | 21.6 |
| Viscosity | 1210 | 480 | 430 | 410 | 1360 | 640 | 580 | 370 |
| Tensile | 1690 | 1240 | 1260 | 1430 | 1490 | 1410 | 1390 | 1340 |
| Tear | 1330 | 840 | 660 | 750 | 1580 | 720 | 690 | 770 |
| Brightness | 28.8 | 28.7 | 24.0 | 29.5 | 25.6 | 28.4 | 29.1 | 29.5 |
| Opacity | 94.6 | 95.3 | 93.8 | 95.1 | 95.2 | 95.1 | 94.5 | 94.6 |

As shown in Tables VI and VII below, treatment of pulp with decay fungi 24P has less of an effect on disaggregated pulp than on pressed pulp. In general, the highest delignification coupled with the smallest decrease in mechanical properties occured at 25 days of treatment.

TABLE VI
Effect of treatment with decay fungi 24P on properties of pressed
pulp

| | Kappa 35 | | | | Kappa 42 | | | |
|---|---|---|---|---|---|---|---|---|
| | Control | 15 days | 25 | 35 | Control | 15 days | 25 | 35 |
| Kappa | 35 | 30.2 | 30.3 | 31.0 | 42 | 36.6 | 31.8 | 33.5 |
| Viscosity | 1510 | 480 | 470 | 420 | 2260 | 580 | 510 | 440 |
| Tensile | 2420 | 1450 | 1620 | 1580 | 1820 | 1190 | 1400 | 1380 |
| Tear | 1030 | 760 | 830 | 740 | 740 | 690 | 720 | 640 |
| Brightness | 27.1 | 25.8 | 23.7 | 24.7 | 23.6 | 22.9 | 22.1 | 22.9 |
| Opacity | 92.6 | 94.1 | 93.6 | 92.9 | 93.2 | 94.3 | 94.0 | 94.2 |

TABLE VII
Effect of treatment with decay fungi 24P on properties of
disggregated pulp

| | Kappa 28 | | | | Kappa 32 | | | |
|---|---|---|---|---|---|---|---|---|
| | Control | 15 days | 25 | 35 | Control | 15 days | 25 | 35 |
| Kappa | 28 | 22.8 | 24.9 | 26.7 | 32 | 30.5 | 29.2 | 29.7 |
| Viscosity | 1210 | 460 | 410 | 390 | 1360 | 840 | 790 | 720 |
| Tensile | 1690 | 1450 | 1540 | 1580 | 1490 | 1320 | 1300 | 1290 |
| Tear | 1330 | 870 | 690 | 770 | 1580 | 780 | 750 | 810 |
| Brightness | 28.8 | 27.7 | 27.9 | 28.2 | 25.6 | 26.4 | 26.1 | 26.5 |
| Opacity | 94.6 | 94.9 | 94.8 | 95.0 | 95.2 | 95.3 | 94.9 | 95.2 |

As shown above, treatment of wood with decay fungi results in substantial delignification of the wood, the extent of which can be varied depending on initial lignin content, and the period for which the pulp is exposed to the decay fungi.

### EXAMPLE 4

The following example presents data from an industrial trial conducted at Constitución Mill, Chile.

Decay fungi strain 24P was applied to 960 m³ of Pinus radiata logs at an application rate of 0.6% (w/w) fungi/dry weight of wood.

Treated wood was stored for 85 days under the same conditions as the normal wood supply of the mill. 6 batch cooking processes were performed, and the characteristics of the resulting pulps measured.

A second experiment utilised basquet technology. Wood was separated into six basquets, and 3 basquets were put into each of 2 batch digesters. The 3 basquets were distributed at differentes height within the digester.

The wood was processed using normal RDH kraft cooking.

Table VIII below presents the characteristics of the pulps derived from the decay fungi-treated wood and control pulp derived from untreated wood, and the properties of paper derived from the treated and control pulps.

TABLE VIII

| | Control | Batch digester | Basket |
|---|---|---|---|
| Wood density (kg/m³) | 390 | 388 | 386 |
| Yield (%) | 47.8 | 49.4 | 49.6 |
| Kappa | 34.6 | 31.4 | 31.2 |
| Viscosity (cp) | 117.8 | 128.0 | 131.0 |
| Unbleached paper | | | |
| Refining (kwh/t) | 74 | 58 | 57 |
| Tear (mNm²/g) | 89.4 | 138 | 141 |
| TEA (j/kg) | 1600 | 2040 | 2100 |
| Tensile (kpa) | 77 | 101 | 104 |

data is the mean of 3 repetitions

As can be seen in Table VIII, treatment of wood with decay fungi results in improved pulp yield, decreased kappa, and improved viscosity. The properties of paper derived from pulps themselves derived from decay fungi-treated wood (known herein as “biopulps”) are also improved relative to paper from pulp derived from untreated wood. Tear, TEA and Tensile indices of paper from biopulps are all substantially superior to the corresponding indices for paper derived from untreated pulp. Finally, the pulping efficiency of wood treated with decay fungi is substantially increased relative to untreated wood; total energy consumed during the refining of biopulps was reduced by approximately 22-23% relative to control pulps.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of a Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of isolating decay fungi which will have a positive effect on non-sterilized wood and/or wood products and/or wood processing by providing a lignocellulosic and/or extractives decrease in the wood and a minimisation or inhibition of the detrimental effects of competitor fungi, the method comprising or including the steps of:
1) collection of decay fungi (whether from nature or otherwise);
2) preparation of a cultivation of the decay fungi;
3) subjecting the cultivated decay fungi to a selection process to distinguish desired decay fungi from unwanted fungi, wherein the selection process includes or comprises subjecting the cultivated decay fungi to both:
a) a test to establish production of oxidative enzymes; and
b) a test to establish the ability of the cultivated decay fungi to outgrow and/or inhibit the development of competitor fungi, and wherein the desired decay fungi will satisfy both tests;
4) isolation of the desired decay fungi.

2. The method according to claim 1 wherein the method additionally includes or comprises the step of identifying the decay fungi at any time.

3. The method according to claim 1 wherein the decay fungi has a minimal deleterious effect on the wood and/or wood products and/or wood processing.

4. The method according to claim 3 wherein the decay fungi has a minimal deleterious effect on cellulose yield and/or polymerisation.

5. The method according to claim 1 wherein the collection of step 1) is from nature.

6. The method according to claim 5 wherein the collection of step 1) is from soil and/or humus.

7. The method according to claim 5 wherein the collection of step 1) occurs on wood and/or tree(s).

8. The method according to claim 7 wherein the collection of step 1) occurs on trees and/or wood of the species Pinus radiata and/or on trees and/or wood of Eucalyptus spp.

9. The method according to claim 1 wherein the cultivation of step 3) occurs between particulate wood including wood chips, sawdust or the like as a solid cultivation, or in liquid supplemented growth media as a liquid cultivation, or in a combination of both as a semi-solid cultivation.

10. The method according to claim 9 wherein the particulate wood of the solid and/or semi-solid cultivation is Pinus radiata or Eucalyptus spp.

11. A biologically pure culture of decay fungi which will have a positive effect on non-sterilized wood and/or wood products and/or wood processing by providing a lignocellulosic and/or extractives decrease in the wood and a minimisation or inhibition of the detrimental effects of competitor fungi isolated according to the above method.

12. The culture according to claim 11 wherein the decay fungi are of the class Basidiomycetes, order Aphyllophorales.

13. The culture according to claim 12 wherein the decay fungi are selected from the genera comprising Pleurotus spp., Coriolus spp., Phanerochaete spp., Phlebia spp., Ganoderma spp., and/or Lentinus spp.

14. The culture according to claim 13 wherein the decay fungi is the Pleurotus sp. strain 10-P and/or 24-P and/or the Coriolus sp. strain 15-A.

15. The culture according to claim 13 wherein the decay fungi is a strain of Coriolus versicolor having all the identifying characteristics of the fungi of AGAL Accession Number NM02/32225.

16. The culture according to claim 11 wherein the decay fungi are of the class Ascomycetes, order Plectoascomycetes.

17. A biologically pure culture of decay fungi which when subjected to the steps of:
1) preparation of a cultivation of the decay fungi;
2) subjecting the cultivated decay fungi to a selection process to distinguish desired decay fungi from unwanted fungi, wherein the selection process includes or comprises subjecting the cultivated decay fungi to both:
a) a test to establish production of oxidative enzymes; and
b) a test to establish the ability of the cultivated decay fungi to outgrow and/or inhibit the development of competitor fungi, and wherein the desired decay fungi will satisfy both tests;
3) isolation of the desired decay fungi;
will, when applied to wood, have a positive effect on non-sterilized wood and/or wood products and/or wood processing by providing a lignocellulosic and/or extractives decrease in the wood and a minimisation or inhibition of the detrimental effects of competitor fungi.

18. The culture according to claim 17 wherein the decay fungi has a minimal deleterious effect on the wood and/or wood products and/or wood processing.

19. The culture according to claim 18 wherein the decay fungi has a minimal deleterious effect on cellulose yield and/or polymerisation.

20. A method for the preparation of a composition which will have a positive effect on non-sterilized wood and/or wood products and/or wood processing by providing a lignocellulosic and/or extractives decrease in the wood and a minimisation or inhibition of the detrimental effects of competitor fungi, the method comprising or including the steps of:
a) isolation of decay fungi as disclosed in the above method;
b) preparation of a reproductively viable form of said decay fungi;
c) use of said reproductively viable form of said decay fungi, optionally together with one or more acceptable carriers, diluents, or adjuvants, in the preparation of a composition.

21. The method of claim 20 wherein the isolation step a) and/or the preparation step b) of the method includes the preparation of the decay fungi between solid wood.

22. The method of claim 21 wherein said solid wood comprises raw wood residuals exemplified by but not limited to shavings, sawdust and/or chips.

23. The method of claim 21 wherein the the solid wood is of the same genus and/or species as the wood for pulp production to which the composition is to be subsequently applied.

24. The method of claim 21 wherein the the solid wood is Pinus radiata or Eucalyptus spp.

25. The method of claim 20 wherein the preparation of step b) of a reproductively viable form of the decay fungi is by massive vegetative reproduction.

26. The method of claim 20 wherein the preparation of step b) occurs between particulate wood including wood chips, sawdust or the like as a solid cultivation, or in liquid supplemented growth media as a liquid cultivation, or in a combination of both as a semi-solid cultivation.

27. The method of claim 26 wherein the particulate wood of the solid and/or semi-solid cultivation is Pinus radiata or Eucalyptus spp.

28. The method of claim 20 wherein the carrier is H₂O.

29. A composition comprising decay fungi which will have a positive effect on non-sterilized wood and/or wood products and/or wood processing by providing a lignocellulosic and/or extractives decrease in the wood and a minimisation or inhibition of the detrimental effects of competitor fungi, prepared according to the above method.

30. The composition according to claim 29 wherein the composition is liquid.

31. The composition according to claim 29 wherein the composition is solid.

32. The composition according to claim 29 wherein the decay fungi has a minimal deleterious effect on the wood and/or wood products and/or wood processing.

33. The composition according to claim 32 wherein the decay fungi has a minimal deleterious effect on cellulose yield and/or polymerisation.

34. The composition according to claim 29 wherein the decay fungi is the Pleurotus sp. strain 10-P and/or 24-P and/or the Coriolus sp. strain 15-A.

35. The composition according to claim 29 wherein the decay fungi is, or the composition includes, Coriolus versicolor AGAL Accession Number NM02/32225.

36. A method of enhancing wood or wood products quality, the method comprising or including the steps of:
a) preparation of a composition comprising or including decay fungi according to the above method;
b) application of the composition to non-sterilized wood subsequently used for pulp production.

37. The method according to claim 36 wherein the application of step b) of the composition is manual and/or automated.

38. The method according to claim 36 wherein the application in step b) of the composition is to non-sterilized wood in the forest and/or storing yard and/or mill.

39. The method according to claim 36 wherein the application in step b) of the composition is to non-sterilized wood at a ratio of between about 0.05% and about 5% w/w) decay fungi/dry weight of wood.

40. The method according to claim 36 wherein the application in step b) of the composition is to non-sterilized wood which has a moisture content of from about 60% to about 80%.

41. The method according to claim 36 wherein the application in step b) of the composition is to non-sterilized wood which comprises logs with or without bark and/or chips.

42. The method according to claim 36 wherein the method includes the step of maintaining the wood to which the composition has been applied under conditions which allow growth of the decay fungi for a term sufficient to allow a minimisation or inhibition of the detrimental effects of competitor fungi.

43. The method according to claim 36 wherein the method includes the step of maintaining the wood to which the composition has been applied under conditions which allow growth of the decay fungi for a term sufficient to effect a lignocellulosic and/or extractives decrease in said wood.

44. The method according to claim 36 wherein in step b) the composition is applied so as to be in contact with the non-sterilized wood for a period of from about 4 days to about 4 months.

45. The method according to claim 44 wherein in step b) the composition is applied to wood chips so as to be in contact with wood for a period of about 7 days.

46. The method according to claim 36 wherein in step b) the composition is applied so that greater than 50% of the wood is colonized by the decay fungi.

47. The method according to claim 36 wherein the decay fungi is, or the composition includes, Pleurotus sp. strain 10-P and/or 24-P and/or the Coriolus sp. strain 15-A.

48. The method according to claim 36 wherein the decay fungi is, or the composition includes, Coriolus versicolor AGAL Accession Number NM02/32225.

49. The wood or wood products prepared according to the method of claim 36.

50. A method of improved chemical and/or modified chemical pulping, the method comprising or including:
a) preparing a composition comprising or including decay fungi able to outgrow competitor fungi on non-sterilized wood;
b) applying the composition to non-sterilized wood to be used for pulp production; and
c) pulping said wood in a chemical and/or modified chemical pulping process.

51. The method according to claim 50 wherein the pulping of step c) is in a kraft and/or modified kraft process.

52. The method according to claim 50 wherein the preparation of step a) of a composition comprising or including decay fungi is by a method according to claim 20.

53. The method according to claim 50 wherein the application of step b) of the composition provides one or more of an increase in pulping efficiency, increased yield, and/or lower kappa numbers.

54. The method according to claim 50 wherein the application of step b) of the composition provides a reduction in pulping energy consumption.

55. The method according to claim 50 wherein the application of step b) of the composition provides a reduction in pulping chemical processing liquour consumption.

56. The method according to claim 50 wherein the application of step b) of the composition is manual and/or automated.

57. The method according to claim 50 wherein the application in step b) of the composition is to non-sterilized wood in the forest and/or storing yard and/or mill.

58. The method according to claim 50 wherein the application in step b) of the composition is to non-sterilized wood at a ratio of between about 0.05% and about 5% (w/w) decay fungi/dry weight of wood.

59. The method according to claim 50 wherein the application in step b) of the composition is to non-sterilized wood which has a moisture content of from about 60% to about 80%.

60. The method according to claim 50 wherein the application in step b) of the composition is to non-sterilized wood which comprises logs with or without bark and/or chips.

61. The method according to claim 50 wherein the method includes the step of maintaining the wood to which the composition has been applied under conditions which allow growth of the decay fungi for a term sufficient to allow a minimisation or inhibition of the detrimental effects of competitor fungi.

62. The method according to claim 50 wherein the method includes the step of maintaining the wood to which the composition has been applied under conditions which allow growth of the decay fungi for a term sufficient to effect a lignocellulosic and/or extractives decrease in said wood.

63. The method according to claim 50 wherein in step b) the composition is applied so as to be in contact with the non-sterilized wood for a period of from about 4 days to about 4 months.

64. The method according to claim 63 wherein in step b) the composition is applied to wood chips so as to be in contact with wood for a period of about 7 days.

65. The method according to claim 50 wherein in step b) the composition is applied so that greater than 50% of the wood is colonized by the decay fungi.

66. The method according to claim 50 wherein the decay fungi is, or the composition includes, Pleurotus sp. strain 10-P and/or 24-P and/or the Coriolus sp. strain 15-A.

67. The method according to claim 50 wherein the decay fungi is, or the composition includes, Coriolus versicolor AGAL Accession Number NM02/32225.

68. A pulp prepared according to claim 50.

69. A biologically pure culture of Coriolus versicolor AGAL Accession Number NM02/32225.

70. The use of Coriolus vesicular AGAL Accession Number NM02/32225 in a composition according to claim 29.

71. The use of Coriolus vesicular AGAL Accession Number NM02/32225 in a method according to claim 36.

72. The use of Coriolus vesicular AGAL Accession Number NM02/32225 in a method according to claim 50.
